(19) United States
(12) Patent Application Publication
(10) Pub. No.: US 2007/0031357 A1
(43) Pub. Date: Feb. 8, 2007

(54) High PH compositions

(76) Inventors: Anton Mentlik, Grapevine, TX (US); Louis Bernard Fisher, Dallas, TX (US)

Correspondence Address: FULBRIGHT & JAWORSKI L.L.P., 600 CONGRESS AVE., SUITE 2400, AUSTIN, TX, 78701, US

(21) Appl. No.: 11/492,670

(22) Filed: Jul. 25, 2006

Related U.S. Application Data
(60) Provisional application No. 60/704,652, filed on Aug. 2, 2005.

(51) Int. Cl. A61K 8/35

(52) U.S. Cl. 424/59

(57) ABSTRACT

Disclosed is a method for tanning skin comprising applying to the skin a sunless tanning composition comprising dihydroxyacetone (DHA) and a high pH composition, where the high pH composition is applied to the skin after the sunless tanning composition is applied to the skin. The high pH composition can increase the effectiveness of the sunless tanning composition by increasing the duration of a tan obtained by using the sunless tanning composition.

### CROSS-REFERENCES TO RELATED APPLICATIONS

This application claims the benefit of U.S. Provisional Application No. 60/704,652, filed Aug. 2, 2005, the contents of which are incorporated by reference.

### BACKGROUND OF THE INVENTION

A. Field of the Invention

The present invention relates generally to high pH compositions and methods for their use in skin-tanning applications and for improving the skin's visual appearance. In certain aspects, the high pH compositions can be used to extend the duration of a sunless tan or improve the effectiveness of a sunless skin tanning product. In other aspects, the high pH compositions can be used to improve the skin's visual appearance by preventing or treating aged or environmentally damaged skin.

B. Background of the Invention
1. Sunless Tanners

Enjoying the sun has been a favorite pastime for people of all ages for many years. A product of enjoying the sun is obtaining a tan. Many societies associate darkened or tanned skin with health and beauty. Unfortunately, sun exposure can be damaging to the skin and has been shown to cause wrinkles, brown age spots, blotchiness, and leathery, sagging skin. In worst-case scenarios, over-exposure to the sun can cause skin cancer which can be disfiguring and even deadly.

Sunless tanning agents can be used to obtain a tan without the need to expose skin to the damaging rays of the sun. For instance, products containing dihydroxyacetone (“DHA”) have been marketed for several decades as being able to darken the skin without the need for sun exposure. DHA is a colorless sugar that interacts with dead skin cells in the stratum corneum of the epidermis. The skin cells change color because of this interaction thereby giving the appearance of a sun tan. The color change typically lasts for about 5 to 7 days and will gradually fade.

To maintain such a tan, a user typically has to reapply the sunless tanning product every four to five days. Reapplication at such short intervals can be time-consuming and is often inconvenient. For instance, in order to apply a sunless tanning product the user typically bathes, exfoliates, and dries the skin prior to application. During the application process, an even application of the sunless tanning product is typically desired to avoid an un-even or “streaky” looking tan. After application, the user typically has to wait for the product to dry or set into the skin. Further, the costs associated with obtaining a sunless tan increase as the use of the product increases.
2. Aged or Environmentally Damaged Skin

With chronological age, chronic exposure to adverse environmental factors, or malnutrition, the visual appearance, physical properties, and physiological functions of skin change in ways that are considered cosmetically undesirable. The most notable and obvious changes include the development of fine lines and wrinkles, loss of elasticity, increased sagging, loss of firmness, loss of skin clarity or color evenness, coarse surface texture, and mottled pigmentation. Many of the alterations in appearance and function are caused by changes in the outer epidermal layer of the skin, while others are caused by changes in the lower dermis.

Several different approaches have been used to treat aged or environmentally-damaged skin, or skin that is unhealthy due to malnutrition. One approach involves the use of specific agents to directly stimulate or inhibit selected biochemical targets. Examples include the use of retinoids to stimulate collagen and glycosaminoglycan synthesis by fibroblasts (Schiltz et al., 1986). Another approach is to use agents or processes that stimulate the rate at which the epidermis replaces itself, a process known as epidermal cell renewal. Increases in epidermal cell renewal rates usually result from a more rapid rate of replication of epidermal basal cells, and can be caused by diverse stimuli such as chemical or physical injury, adverse environmental conditions, or direct stimulators of basal cell division.

Agents that directly or indirectly stimulate basal cell division include hydroxy acids, retinoids, or barrier disrupters. For example, U.S. Pat. No. 5,720,963 discloses that a combination of hydroxy acids, retinoids, and cerebrosides causes chronic injury to the stratum corneum and results in epidermal and dermal repair of the structurally-deteriorated skin. U.S. Pat. No. 6,495,126, for example, uses a combination of surfactants and chelating agents to stimulate an endogenous stratum corneum chymotryptic proteinase that causes a loosening of corneocytes, resulting in an increased rate of epidermal replacement and chronic anti-aging benefits. Adverse environmental exposures that can result in more rapid epidermal turnover rates include UVA, UVB, and IR radiation from the sun and cold coupled with low relative humidity (i.e. low dew point).

Many of the above methods of increasing stratum corneum renewal rates have various drawbacks, such as significant irritation to the skin, skin toxicity, or low pH (e.g., alpha-hydroxy acids). In addition, most of these methods involve the invocation of chronic damage to the skin, which sets up repair mechanisms. For most of the existing treatments, there will be a period of time, up to several weeks or months, during which the skin becomes irritated and after which tolerance sets in and the symptoms of irritation may decrease and/or cease.

### SUMMARY OF THE INVENTION

The present invention overcomes the deficiencies in the art by providing compositions and methods for their use in sunless tanning applications. The compositions can also be used to treat or prevent aged, mature, nutritionally-compromised, or environmentally-damaged skin.

In one aspect of the present invention, there is disclosed a method for tanning skin comprising applying to the skin a sunless tanning agent and a high pH composition. The use of a sunless tanning agent with a high pH composition can extend the duration or time of a sunless tan. The increase in duration can be determined, for example, by measuring or calculating the duration of a sunless tan that was obtained by using a sunless tanning agent and a high pH composition, and then measuring or calculating the duration of a sunless tan that was obtained by using a sunless tanning agent and no high pH composition. The difference in the durations can show the increased amount of time that a sunless tan lasts when using a sunless tanning agent and a high pH composition to obtain the tan. By way of example only, the inventors discovered that a sunless tanning product used with a high pH composition can produce a sunless tan that lasts for at least about 5, 6, 7, 8, 9, 10, 11, 12, 13, 14, 15, 16, 17, 18, 19, 20, 21, or more days. In other non-limiting aspects, the high pH composition can be applied directly onto the site that the sunless tanning composition was applied to the skin. In other embodiments, the high pH composition can be applied to the skin at a site that is different to the site that the sunless tanning composition was applied to the skin.

The sunless tanning agent and the high pH composition can be included in a single composition or in separate compositions. In instances where the sunless tanning agent and the high pH composition are included in separate compositions, the separate compositions can be included in separate chambers or compartments of a bottle, dispenser, or container. In other instances, the separate compositions can be included in separate bottles, dispensers, or containers.

In other non-limiting embodiments, the high pH composition can be applied to the skin before, at the same time as, or after applying the sunless tanning agent. For example, the high pH composition can applied to the skin at about 5, 10, 15, 20, 25, 30, 35, 40, 45, 50, or 55 seconds before, or 1, 2, 3, 4, 5, 6, 7, 8, 9, 10, 15, 20, 25, 30, 35, 40, 45, 50, 55, 60, or 90 minutes before, or 2, 3, 4, 5, 6, 7, 8, 9, 10, 11, 12, 15, 18, 24, 25, 26, 27, 28, 29, 30, 31, 32, 33, 34, 35, 36, 37, 38, 39, 40, 41, 42, 43, 44, 45, 46, 47, or 48 hours before, or 3, 4, 5, 6, 7, 8, 9, 10, 11, 12, 13, or 14 days before the sunless tanning agent. In other aspects, the high pH composition can be applied to the skin at about 5, 10, 15, 20, 25, 30, 35, 40, 45, 50, or 55 seconds after, or 1, 2, 3, 4, 5, 6, 7, 8, 9, 10, 15, 20, 25, 30, 35, 40, 45, 50, 55, 60, or 90 minutes after, or 2, 3, 4, 5, 6, 7, 8, 9, 10, 11, 12, 15, 18, 24, or 48 hours after, or 3, 4, 5, 6, 7, 8, 9, 10, 11, 12, 13, or 14 days after the sunless tanning agent. In certain aspects of the present invention, obtaining and/or maintaining a sunless tan includes following a regimen. For example, the regimen can include applying the sunless tanning agent and a high pH composition in a first instance as disclosed throughout this specification. The regiment can then include additional applications that are identical or similar to the first instance. The additional applications can include, for example, a second, third, fourth, fifth, sixth, seventh, eighth, nine, tenth, or more instances to obtain and/or maintain a sunless tan.

The sunless tanning agent, in other non-limiting aspects of the present invention, can be comprised in a sunless tanning composition, formulation, or product. The product can be formulated, for example, as a spray, mouse, gel, foam, cream, liquid, or powder. Non-limiting examples of sunless tanning products include: (i) Schering-Plough's “Bain de Soleil Streakguarde Mouse, Tinted Tanning Foam,” “Bain de Soleil Streakguarde Self Tanning Crème,” “Bain de Soleil Mega Tan Sunscreen Lotion with Self Tanner,” “Coppertone Oil Free Sunless Tanning Lotion”; (ii) Playtex Products, Inc.'s “Banana Boat Sunless Tanning Crème”; (iii) Neutrogena's “Sunless Tanning Foam” and “Build-A-Tan Gradual Sunless Tanning”; and (iv) SkinCeuticals “Sunless Tanning Lotion.” This list of products are exemplary only, and it is contemplated by the inventors that any sunless tanning product can be used by the present invention. In certain embodiments, sunless tanning products that include DHA are preferred.

In other non-limiting aspects of the present invention, the sunless tanning agent can include bronzers, pigmentation agents (e.g., methoxsalen, trioxsalen, and melanin), dyes, botanical extracts (e.g., silver birch (Betulla alba), and Mahakanni STLC (Eclipta alba)), and chemical compounds (e.g. dihydroxyacetone, erythrulose, lawsone, tyrosine, orjugulone, alpha-hydroxy aldehydes and ketones, glyceraldehyde and related alcohol aldehydes, various indoles, imidazoles, methyl glyoxal, glycerol aldehyde, erythrulose, alloxan, 2,3-dihydroxysuccindialdehyde, 2,3-dimethoxysuccindialdehyde, 2-amino-3-hydroxy-succindialdehyde and 2-benzylamino-3-hydroxysuccindialdehyde). Other sunless tanning agents, including those known and unknown to a person of skill in the art, are also contemplated as being useful with the present invention.

The high pH compositions of the present invention, in non-limiting aspects, can have a pH of about 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, or 14.0, or more. In certain preferred aspects, the pH of the compositions of the present invention will be in the range of about 7.5 to about 9.5.

In non-limiting embodiments, the high pH compositions of the present invention can include a fatty acid. The fatty acid can be a saturated, mono-unsaturated, poly-unsaturated fatty acid, straight chain, and/or branched for example. Non-limiting examples of fatty acids include C2-C34 fatty acids, and preferably C6-C23 straight chain and branched fatty acids (e.g., oleic acid, stearic acid, caproic acid, or behenic acid)). The high pH compositions can also include an inorganic and/or organic base (non-limiting examples include NaOH, KOH, and triethanol amine). The high pH compositions can also include an emollient (non-limiting example is a mixture of cosmetic esters and/or oils), a film former (used to keep or retain moisture in the skin (e.g., C10-C30 cholesterols) (non-limiting examples include cellulosics, natural gums, alginates, natural and modified clays, acrylic film formers, petrolatum, petroleum derived film formers (non-limiting examples are hydrogenated isobutenes)) and other known to those of skill in the art, a buffer, a penetrant (non-limiting example includes oleic acid, glycol ethers, low molecular weight alcohols, and DMSO), a hydroxide (non-limiting examples include Na, K, Ca, NH₄, Mg, Li, organic amines such as triethanol amines, Quadrol, AMPD, and amine oxides), an amine, an emulsifier (non-limiting example includes ionic, zwitterionic, or non-ionic emulsifies such as neutral fatty acids and esters of the same, nonionic sorbitol esters, ethoxylated fatty alcohols and esters thereof, alkyl sulfates, sulfonates, and polymeric emulsifiers), and/or a humectant (non-limiting examples include glycerin and its ethoxylated derivatives, glycols from propylene to pentalyne, and polyethylene glycols). In other non-limiting aspects, the high pH compositions of the present invention do not include a hydroxy acid (e.g., alpha-hydroxy acid). In certain aspects, the high pH composition is not a cleansing composition (e.g., soap) or a moisturizer.

The sunless tanning agent and/or the high pH compositions of the present invention can be included in a cosmetic vehicle. The cosmetic vehicle can include an emulsion (e.g., water-in-oil or oil-in-water), a cream, a lotion, a solution, an anhydrous base, a gel, or an ointment. The solution, for example, can be an aqueous solution or hydro-alcoholic solution. The high pH composition can be included in an anti-aging product, cleansing product, or a moisturizing product, for example. It is contemplated, however, that the high pH compositions can be included in other products discussed throughout this specification and those known to a person of ordinary skill in the art. The sunless tanning and/or high pH compositions of the present invention can also be formulated or adapted to be applied at least 1, 2, 3, 4, 5, 6, 7, 8, 9 or more times a day.

In another aspect of the present invention, there is provided a kit for artificially tanning skin comprising a sunless tanning agent and a high pH composition. The sunless tanning agent and the high pH composition can be included in separate containers (e.g., bottles, packages, or dispenser). Alternatively, the sunless tanning agent and the high pH composition can be included in the same container as a single composition or as separate compositions. In instances where the sunless tanning agent and the high pH composition are separate compositions, the separate compositions can be included in separate chambers or compartments of a container. The kits of the present invention can also include instructions. The instructions can include an explanation of how to apply, use, and maintain the products or compositions, for example. In other instances, the kit may have indicia on their outer or inner surfaces. The indicia may be a writing, image, picture, or inscription. The indicia, by way of example only, may recite “Mary Kay,” “Sunless Tanner,” “Anti-Aging,” etc.

In yet another embodiment of the present invention, there is disclosed a method for extending the duration of a sunless tan comprising applying to the skin a sunless tanning agent and a high pH composition. There is also disclosed a method for increasing the effectiveness of a sunless tanning product comprising applying to skin a sunless tanning product and a high pH composition. The effectiveness of the sunless tanning product is increased by increasing the duration of a tan obtained by using the sunless tanning product. As noted above, the inventors contemplate that any sunless tanning product can be used with the present invention. In certain embodiments, sunless tanning products that include DHA are preferred.

A further embodiment of the present invention includes a method of increasing the efficacy of a sunless skin tanning product or sunless skin tanning agent comprising applying a sunless tanning agent or product to the skin and subsequently applying a high pH composition to the skin. In certain embodiments, the high pH composition can increase the color of the sunless tan that was created by the sunless tanning agent, thereby increasing skin color. Increasing the color of the sunless tan or increasing skin color can include, for example, making the skin have a darker appearance or a more intense or deeper color tone when compared to a sunless tan obtained by using a skin tanning agent and not subsequently using a high pH composition.

Another aspect of the present invention includes a method of treating or preventing aged or damaged skin comprising topical application of a high pH composition. The high pH composition can be a cosmetically acceptable formulation. The damaged skin can include, for example, nutritionally compromised skin or environmentally damaged skin. In certain aspects, the environmentally damaged skin includes skin damaged by u.v. light, chronic sun exposure, environmental pollutants, chemicals, disease pathologies, or smoking. As noted above, the high pH compositions can be included in an anti-aging product, cleansing product, or a moisturizing product, for example. In a non-limiting embodiment, the method can further be defined as a method of activating or stimulating epidermal cell division. The activation or stimulating of epidermal cell division can be used to prevent or treat symptoms associated with aged or damaged skin (e.g., fine lines and wrinkles, loss of elasticity, increased sagging, loss of firmness, loss of skin clarity or color evenness, coarse surface texture, and mottled pigmentation).

It is contemplated that any embodiment discussed in this specification can be implemented with respect to any method or composition of the invention, and vice versa. Furthermore, compositions of the invention can be used to achieve methods of the invention.

The terms “inhibiting,” “reducing,” or “prevention,” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.” Throughout this application, the term “about” is used to indicate that a value includes the inherent variation of error for the device, the method being employed to determine the value, or the variation that exists among study subjects.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### DESCRIPTION OF ILLUSTRATIVE EMBODIMENTS

The use of sunless tanning compositions have gained more and more popularity over the years. An advantage of sunless tanning compositions includes the possibility of obtaining a tan without having to expose skin to the damaging sun rays. One of the more widely used tanning agents in sunless tanning products is DHA.

The inventors' discovery of using high pH compositions in combination with sunless tanning products to increase the duration of a sunless tan provides several advantages over the art. For instance, because of the increased duration of the sunless tan, reapplication intervals of the sunless tanning product can be decreased. Stated another way, a person's tan will last for a longer period of time.

The high pH compositions of the present invention can also be used to improve the skin's visual appearance by preventing or treating aged or environmentally damaged skin. For instance, the high pH compositions can be used activate epidermal cell division which can decrease the appearance of aging skin (e.g., dryness, increased texture, staginess, fine lines and wrinkles).

These and other aspects of the present invention are described in further detail in the following sections.

A. High pH Compositions

High pH compositions can irritate human skin causing the skin to become dry and flaky. An increased pH can also cause swelling of the stratum corneum which can result in an increase in water loss from the keratinocytes thereby making them more prone to physical damage. There is a feedback system between keratinocyte loss from the skin surface and cell multiplication at the base layer of the epidermis. Damage caused at the surface of human skin by a high pH composition is followed by an increase in cell division. This increase in cell division will replace the surface cells and increase the thickness of the epidermis which tends to become thinner with age.

Topical skin care products and compositions having a high pH can be used to activate or “turn on” epidermal cell division. This activation can prevent or treat symptoms associated with aged or damaged skin (e.g., fine lines and wrinkles, loss of elasticity, increased sagging, loss of firmness, loss of skin clarity or color evenness, coarse surface texture, and mottled pigmentation).

In certain non-limiting aspect, the high pH compositions of the present invention will have a pH of about 7.5, 8.0, 8.5, 9.0, 9.5, 10.0, 10.5, 11.0, 11.5, 12.0, 12.5, 13.0, 13.5, or 14. A person of ordinary skill in the art would understand how to vary the pH of a particular composition. By way of example only, the pH of a composition can be increased or decreased by the addition or subtraction of H⁺ or OH⁻ ions to the composition.

In other embodiments, the pH compositions of the present invention can also include penetrants. Penetrants can be used to increase the efficacy of a given compositions by delivering the active ingredients deeper into the skin. Non limiting examples of penetrants that can be used with the present invention include oleic acid, glycol ethers, low molecular weight alcohols, dimethyl sulfoxide (DMSO), and polyethylene glycols (e.g., PEG-1, 2, 3, 4, 5, 6, 7, 8, 9, 10, 11, 12, 13, 14, 15, 16, 17, 18, 19, 20, 25, 30, etc.). The high pH compositions of the present invention can also include additional agents and compounds that are disclosed in this specification and known to those of ordinary skill in the art. For example, the compositions can also include moisturizing agents, anti-oxidants, sunscreens having UVA and/or UVB protection, emollients, anti-irritants, vitamins, trace metals, anti-microbial agents, botanical extracts, fragrances, and/or dyes and color ingredients. In preferred embodiments, the high pH compositions can include a fatty acid. In certain embodiments, the fatty acid can be a C2 to C34 fatty acid. In preferred aspects, the fatty acid can be a C12 to C18 fatty acid. In more preferred embodiments, the fatty acid can be a C1 8 unsaturated fatty acid (i.e., oleic acid) or a C18 saturated fatty acid (i.e., stearic acid). The high pH composition can include an inorganic and organic base (e.g., NaOH, KOH, and triethanol amine), an emollient, a film former (used to keep or retain moisture in the skin (e.g., C10-C30 cholesterols), a buffer, a penetrant (e.g. oleic acid), an hydroxide, an amine, an emulsifier (e.g., ionic, zwitterionic, or non-ionic) and/or a humectant. The high pH compositions of the present invention can also preferably cause an increase in cell turnover with minimal skin irritancy.

B. Sunless Tanning Agents

In another non-limiting aspect of the present invention, there are provided methods of tanning skin, extending the duration of a sunless tan, and increasing the effectiveness of a sunless tanning product. Each of these methods can use a sunless tanning agent, or a derivative thereof. For purposes of the present specification, the phrases “sunless tanning agent,” “sunless tanner, “skin tanner, “skin tanning agent, “self tanning agent,” “sunless tanning active,” or “self tanning active” can be used interchangeably. Sunless tanning agents can include any compound or material which can stain skin or cause skin pigmentation to darken by exposure to the sunless tanning agent, without the need to expose the skin to sun or artificial ultraviolet light rays.

Non-limiting examples of sunless tanning agents that can be used with the present invention include bronzers (e.g., creams, liquids, sprays, and powders that a person can apply to skin to make the skin look tan, brown, or darker from being in the sun), pigmentation agents (e.g., methoxsalen, trioxsalen, and melanin), dyes, botanical extracts (e.g., silver birch (Betulla alba), and Mahakanni STLC (Eclipta alba)), and chemical compounds (e.g. dihydroxyacetone, erythrulose, lawsone, tyrosine, orjugulone, alpha-hydroxy aldehydes and ketones, glyceraldehyde and related alcohol aldehydes, various indoles, imidazoles, methyl glyoxal, glycerol aldehyde, erythrulose, alloxan, 2,3-dihydroxysuccindialdehyde, 2,3-dimethoxysuccindialdehyde, 2-amino-3-hydroxy-succindialdehyde and 2-benzylamino-3-hydroxysuccindialdehyde). Other sunless tanning agents, including those known and unknown to a person of skill in the art, are also contemplated as being useful with the present invention. Non-limiting examples of the sunless tanning agents are also described in U.S. Pat. Nos. 6,482,397; 6,262,541; 5,559,146; 6,447,760; and 6,443,164.

In certain non-limiting embodiments, dihydroxyacetone (“DHA”) is a preferred sunless tanning agent. DHA colorless sugar (triose carbohydrate) having the chemical formula C₃H₆0₃. It can be prepared by the mild oxidation of glycerol, for example with hydrogen peroxide and a ferrous salt as a catalyst. The chemical formula for DHA is illustrated below: 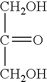
When applied, DHA interacts with dead skin cells located in the stratum corneum of the epidermis. This interaction causes a color change thereby providing a darkening or tanning effect. The color change typically lasts from about five to seven days from the initial application.

In certain aspects, the sunless tanning agents are present in compositions at concentrations at “effective amounts.” Such effective amounts include an amount of the sunless tanning agent which, when applied to skin, causes the skin to darken or tan without the need to expose the skin to natural sunlight or artificial UV light sources. A person of ordinary skill in the art is capable of determining an appropriate amount of the sunless tanning agent in a given composition by using known means (e.g., applying different amounts of particular agents to skin in a testing environment).

C. Source of Compounds, Agents, and Active Ingredients

The compounds, agents, and active ingredients that are described in the claims and specification can be obtained by any means known to a person of ordinary skill in the art. In a non-limiting embodiment, for example, the compounds, agents, and active ingredients can be isolated by obtaining the source of such compounds, agents, and active ingredients. In many instances, the compounds, agents, and active ingredients are commercially available. For example, DHA can be obtained through any number of companies including ScienceLab.com, Inc., located in Kingwood, Tex. (also see www.sciencelab.com), A&E Connock, Girindus Chemie, and Rona/E.M. Industries).

Additionally, the compounds, agents, and active ingredients can be purified by any number of techniques known to a person of ordinary skill in the art. Non-limiting examples of purification techniques include Polyacrylamide Gel Electrophoresis, High Performance Liquid Chromatography (HPLC), Gel chromatography or Molecular Sieve Chromatography, and Affinity Chromatography. In other aspects, the compounds, agents, and active ingredients can be obtained by chemical synthesis or by recombinant means by using conventional techniques. See, for example, Stewart and Young, (1984); Tam et al., (1983); Merrifield, (1986); and Barany and Merrifield (1979), Houghten (1985).

D. Modifications and Derivatives

Modifications or derivatives of the compounds, agents, and active ingredients disclosed throughout this specification are contemplated as being useful with the methods and compositions of the present invention. Derivatives may be prepared and the properties of such derivatives may be assayed for their desired properties by any method known to those of skill in the art.

In certain aspects, “derivative” refers to a chemically modified compound that still retains the desired effects of the compound prior to the chemical modification. Such derivatives may have the addition, removal, or substitution of one or more chemical moieties on the parent molecule. Non limiting examples of the types modifications that can be made to the compounds and structures disclosed throughout this document include the addition or removal of lower alkanes such as methyl, ethyl, propyl, or substituted lower alkanes such as hydroxymethyl or aminomethyl groups; carboxyl groups and carbonyl groups; hydroxyls; nitro, amino, amide, and azo groups; sulfate, sulfonate, sulfono, sulfhydryl, sulfonyl, sulfoxido, phosphate, phosphono, phosphoryl groups, and halide substituents. Additional modifications can include an addition or a deletion of one or more atoms of the atomic framework, for example, substitution of an ethyl by a propyl; substitution of a phenyl by a larger or smaller aromatic group. Alternatively, in a cyclic or bicyclic structure, hetero atoms such as N, S, or O can be substituted into the structure instead of a carbon atom.

E. Equivalents

Known and unknown equivalents to the specific compounds, agents, and active ingredients discussed throughout this specification can be used with the compositions and methods of the present invention. The equivalents can be used as substitutes for the specific compounds, agents, and active components. The equivalents can also be used to add to the methods and compositions of the present invention. A person of ordinary skill in the art would be able to recognize and identify acceptable known and unknown equivalents to the specific compounds, agents, and active ingredients without undue experimentation.

F. Compositions of the Present Invention

A person of ordinary skill would recognize that the compositions of the present invention can include any number of combinations of compounds, agents, and/or active ingredients, or derivatives therein. It is also contemplated that that the concentrations of the compounds, agents, and/or active ingredients can vary. In other non-limiting embodiments, for example, the compositions may include in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 25679330.1 15 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein, of at least one of the compounds, agents, active ingredients, or derivatives that are mentioned throughout the specification and claims. In non-limiting aspects, the percentage can be calculated by weight or volume of the total composition. A person of ordinary skill in the art would understand that the concentrations can vary depending on the addition, substitution, and/or subtraction of the compounds, agents, or active ingredients, to the disclosed methods and compositions.

The disclosed compositions of the present invention may also include various antioxidants to retard oxidation of one or more components. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

G. Cosmetic Vehicles

The compositions of the present invention are effective in all types of cosmetic vehicles. Non-limiting examples of suitable cosmetic vehicles include emulsions, creams, lotions, solutions (both aqueous and hydro-alcoholic), anhydrous bases (such as lipsticks and powders), gels, and ointments or by other method or any combination of the forgoing as would be known to one of ordinary skill in the art (Remington's, 1990). Variations and other appropriate vehicles will be apparent to the skilled artisan and are appropriate for use in the present invention.

In certain aspects, the cosmetic vehicle is selected from oil-in-water emulsions, hydro-alcoholic solutions, or encapsulated beads in anhydrous systems. With respect to oil-in-water emulsions, such emulsions and their compositions and methods of making are well known in the art. It is important, however, that the concentrations and combinations of the compounds and extracts be selected in such a way that the combinations are chemically compatible and do not form complexes which precipitate from the finished product.

H. Cosmetic Products

The composition of the present invention can also be used in many cosmetic products including, but not limited to, sunless skin tanning products, moisturizing creams, skin benefit creams and lotions, softeners, day lotions, gels, ointments, foundations, night creams, lipsticks, cleansers, toners, masks, or other known cosmetic products or applications. Additionally, the cosmetic products can be formulated as leave-on or rinse-off products.

I. Additional Compounds and Agents that can be used in Combination with the Present Compositions

Compositions of the present invention can include other beneficial agents and compounds such as, for example, acute or chronic moisturizing agents (including, e.g., humectants, occlusive agents, and agents that affect the natural moisturization mechanisms of the skin), anti-oxidants, sunscreens having UVA and/or UVB protection, emollients, anti-irritants, vitamins, trace metals, anti-microbial agents, botanical extracts, fragrances, and/or dyes and color ingredients.
1. Moisturizing Agents

Non-limiting examples of moisturizing agents that can be used with the compositions of the present invention include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol.

Other examples include acetylated lanolin, acetylated lanolin alcohol, acrylates/C10-30 alkyl acrylate crosspolymer, acrylates copolymer, alanine, algae extract, aloe barbadensis, aloe-barbadensis extract, aloe barbadensis gel, althea officinalis extract, aluminum starch octenylsuccinate, aluminum stearate, apricot (prunus armeniaca) kernel oil, arginine, arginine aspartate, arnica montana extract, ascorbic acid, ascorbyl palmitate, aspartic acid, avocado (persea gratissima) oil, barium sulfate, barrier sphingolipids, butyl alcohol, beeswax, behenyl alcohol, beta-sitosterol, BHT, birch (betula alba) bark extract, borage (borago officinalis) extract, 2-bromo-2-nitropropane-1,3-diol, butcherbroom (ruscus aculeatus) extract, butylene glycol, calendula officinalis extract, calendula officinalis oil, candelilla (euphorbia cerifera) wax, canola oil, caprylic/capric triglyceride, cardamon (elettaria cardamomum) oil, carnauba (copernicia cerifera) wax, carrageenan (chondrus crispus), carrot (daucus carota sativa) oil, castor (ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (anthemis nobilis) oil, cholesterol, cholesterol esters, cholesteryl hydroxystearate, citric acid, clary (salvia sclarea) oil, cocoa (theobroma cacao) butter, coco-caprylate/caprate, coconut (cocos nucifera) oil, collagen, collagen amino acids, corn (zea mays) oil, fatty acids, decyl oleate, dextrin, diazolidinyl urea, dimethicone copolyol, dimethiconol, dioctyl adipate, dioctyl succinate, dipentaerythrityl hexacaprylate/hexacaprate, DMDM hydantoin, DNA, erythritol, ethoxydiglycol, ethyl linoleate, eucalyptus globulus oil, evening primrose (oenothera biennis) oil, fatty acids, tructose, gelatin, geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (vitis vinifera) seed oil, hazel (corylus americana) nut oil, hazel (corylus avellana) nut oil, hexylene glycol, honey, hyaluronic acid, hybrid safflower (carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, imidazolidinyl urea, iodopropynyl butylcarbamate, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (jasminum officinale) oil, jojoba (buxus chinensis) oil, kelp, kukui (aleurites moluccana) nut oil, lactamide MEA, laneth-16, laneth-10 acetate, lanolin, lanolin acid, lanolin alcohol, lanolin oil, lanolin wax, lavender (lavandula angustifolia) oil, lecithin, lemon (citrus medica limonum) oil, linoleic acid, linolenic acid, macadamia ternifolia nut oil, magnesium stearate, magnesium sulfate, maltitol, matricaria (chamomilla recutita) oil, methyl glucose sesquistearate, methylsilanol PCA, microcrystalline wax, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (olea europaea) oil, orange (citrus aurantium dulcis) oil, palm (elaeis guineensis) oil, palmitic acid, pantethine, panthenol, panthenyl ethyl ether, paraffin, PCA, peach (prunus persica) kernel oil, peanut (arachis hypogaea) oil, PEG-8 C12-18 ester, PEG-15 cocamine, PEG-1 50 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquatemium-24, polysorbate 20, polysorbate 40, polysorbate 60, polysorbate 80, polysorbate 85, potassium myristate, potassium palmitate, potassium sorbate, potassium stearate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, quaternium-15, quaternium-18 hectorite, quaternium-22, retinol, retinyl palmitate, rice (oryza sativa) bran oil, RNA, rosemary (rosmarinus officinalis) oil, rose oil, safflower (carthamus tinctorius) oil, sage (salvia officinalis) oil, salicylic acid, sandalwood (santalum album) oil, serine, serum protein, sesame (sesamum indicum) oil, shea butter (butyrospermum parkii), silk powder, sodium chondroitin sulfate, sodium hyaluronate, sodium lactate, sodium palmitate, sodium PCA, sodium polyglutamate, sodium stearate, soluble collagen, sorbic acid, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (triticum vulgare) germ oil, and ylang ylang (cananga odorata) oil.
2. Antioxidants

Non-limiting examples of antioxidants that can be used with the compositions of the present invention include acetyl cysteine, ascorbic acid, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCI, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.
3. Compounds Having Ultraviolet Light Absorbing Properties

Non-limiting examples of compounds that have ultraviolet light absorbing properties that can be used with the compounds of the present invention include benzophenone, benzophenone- 1, benzophenone-2, benzophenone-3, benzophenone-4 benzophenone-5, benzophenone-6, benzophenone-7, benzophenone-8, benzophenone-9, benzophenone- 10, benzophenone- 11, benzophenone- 12, benzyl salicylate, butyl PABA, cinnamate esters, cinoxate, DEA-methoxycinnamate, diisopropyl methyl cinnamate, ethyl dihydroxypropyl PABA, ethyl diisopropylcinnamate, ethyl methoxycinnamate, ethyl PABA, ethyl urocanate, glyceryl octanoate dimethoxycinnamate, glyceryl PABA, glycol salicylate, homosalate, isoamyl p-methoxycinnamate, PABA, PABA esters, Parsol 1789, isopropylbenzyl salicylate, and octyl methoxycinnamate.
4. Preservatives

Non-limiting examples of preservatives that may used with compositions of the invention include phenonip™, and/or any of its constituents phenoxyethanol, methylparaben, butylparaben, ethylparaben, propylparaben, additionally Suttocide, Germaben™, LiquiPar potassium sorbate, and/or rosemary oleoresin may be used.
5. Structuring Agents

In other non-limiting aspects, the compositions of the present invention can include a structuring agent. Structuring agent, in certain aspects, assist in providing rheological characteristics to the composition to contribute to the composition's stability. In other aspects, structuring agents can also function as an emulsifier or surfactant. Non-limiting examples of structuring agents include stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof. Other non-limiting examples can be found in International Cosmetic Ingredient Dictionary, 10th edition, 2004, which is incorporated by reference.
6. Emulsiflers

In certain preferred aspects of the present invention, the compositions do not include an emulsifier. In other aspects, however, the compositions can include one or more emulsifiers. Emulsifiers can reduce the in interfacial tension between phases and improve the formulation and stability of an emulsion. The emulsifiers can be nonionic, cationic, anionic, and zwitterionic emulsifiers (See McCutcheon's (1986); U.S. Pat. Nos. 5,011,681; 4,421,769; 3,755,560). Non-limiting examples include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates , polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, and mixtures thereof. Other non-limiting examples can be found in International Cosmetic Ingredient Dictionary, 10th edition, 2004, which is incorporated by reference.
7. Silicone Containing Compounds

In non-limiting aspects, silicone containing compounds include any member of a family of polymeric products whose molecular backbone is made up of alternating silicon and oxygen atoms with side groups attached to the silicon atoms. By varying the —Si—O-chain lengths, side groups, and crosslinking, silicones can be synthesized into a wide variety of materials. They can vary in consistency from liquid to gel to solids.

The silicone containing compounds that can be used in the context of the present invention include those described in this specification or those known to a person of ordinary skill in the art. Non-limiting examples include silicone oils (e.g., volatile and non-volatile oils), gels, and solids. In preferred aspects, the silicon containing compounds includes a silicone oils such as a polyorganosiloxane. Non-limiting examples of polyorganosiloxanes include dimethicone, cyclomethicone, polysilicone- b 11, phenyl trimethicone, trimethylsilylamodimethicone, stearoxytrimethylsilane, or mixtures of these and other organosiloxane materials in any given ratio in order to achieve the desired consistency and application characteristics depending upon the intended application (e.g., to a particular area such as the skin, hair, or eyes). A “volatile silicone oil” includes a silicone oil have a low heat of vaporization, i.e. normally less than about 50 cal per gram of silicone oil. Non-limiting examples of volatile silicone oils include: cyclomethicones such as Dow Corning 344 Fluid, Dow Corning 345 Fluid, Dow Corning 244 Fluid, and Dow Corning 245 Fluid, Volatile Silicon 7207 (Union Carbide Corp., Danbury, Conn.); low viscosity dimethicones, i.e. dimethicones having a viscosity of about 50 cst or less (e.g., dimethicones such as Dow Corning 200-0.5 cst Fluid). The Dow Corning Fluids are available from Dow Corning Corporation, Midland, Mich. Cyclomethicone and dimethicone are described in International Cosmetic Ingredient Dictionary, 10th edition, 2004, which is incorporated by reference as cyclic dimethyl polysiloxane compounds and a mixture of fully methylated linear siloxane polymers end-blocked with trimethylsiloxy units, respectively. Other non-limiting volatile silicone oils that can be used in the context of the present invention include those available from General Electric Co., Silicone Products Div., Waterford, N.Y. and SWS Silicones Div. of Stauffer Chemical Co., Adrian, Mich. and those described in International Cosmetic Ingredient Dictionary, 10th edition, 2004.
8. Essential Oils

Essential oils include oils derived from herbs, flowers, trees, and other plants. Such oils are typically present as tiny droplets between the plant's cells, and can be extracted by several method known to those of skill in the art (e.g., steam distilled, enfleurage (i.e., extraction by using fat), maceration, solvent extraction, or mechanical pressing). When these types of oils are exposed to air they tend to evaporate (i.e., a volatile oil). As a result, many essential oils are colorless, but with age they can oxidize and become darker. Essential oils are insoluble in water and are soluble in alcohol, ether, fixed oils (vegetal), and other organic solvents. Typical physical characteristics found in essential oils include boiling points that vary from about 160° to 240° C. and densities ranging from about 0.759 to about 1.096.

Essential oils typically are named by the plant from which the oil is found. For example, rose oil or peppermint oil are derived from rose or peppermint plants, respectively. Non-limiting examples of essential oils that can be used in the context of the present invention include sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang ylang. Other essential oils known to those of skill in the art are also contemplated as being useful within the context of the present invention.
9. Thickening Agents

Thickening agents, including thickener or gelling agents, include substances which that can increase the viscosity of a composition. Preferred thickeners includes those that can increase the viscosity of a composition without substantially modifying the efficacy of the active ingredient within the composition. Thickeners can also increase the stability of the compositions of the present invention.

Non-limiting examples of thickening agents that can be used in the context of the present invention include hydrogenated polyisobutene or trihydroxystearin or combination of both. Other examples include carboxylic acid polymers, crosslinked polyacrylate polymers, polyacrylamide polymers, polysaccharides, and gums. Examples of carboxylic ac id polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see U.S. Pat. Nos. 5,087,445; 4,509,949; 2,798,053; CTFA International Cosmetic Ingredient Dictionary, Tenth Edition, 2004). Examples of commercially available carboxylic acid polymers include carbomers, which are homopolymers of acrylic acid crosslinked with allyl ethers of sucrose or pentaerytritol (e.g., Carbopol™ 900 series from B. F. Goodrich).

Non-limiting examples of crosslinked polyacrylate polymers include cationic and nonionic polymers. Examples are described in U.S. Pat. Nos. 5,100,660 ; 4,849,484; 4,835,206; 4,628,078; 4,599,379).

Non-limiting examples of polyacrylamide polymers (including nonionic polyacrylamide polymers including substituted branched or unbranched polymers) include polyacrylamide, isoparaffin and laureth-7, multi-block copolymers of acrylamides and substituted acrylamides with acrylic acids and substituted acrylic acids.

Non-limiting examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Another example is an alkyl substituted cellulose where the hydroxy groups of the cellulose polymer is hydroxyalkylated (preferably hydroxy ethylated or hydroxypropylated) to form a hydroxyalkylated cellulose which is then further modified with a C10 -C30 straight chain or branched chain alkyl group through an ether linkage. Typically these polymers are ethers of C10-C30 straight or branched chain alcohols with hydroxyalkylcelluloses. Other useful polysaccharides include scleroglucans comprising a linear chain of (1-3) linked glucose units with a (1-6) linked glucose every three unit.

Non-limiting examples of gums that can be used with the present invention include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof.
10. Additional Compounds and Agents

Non-limiting examples of additional compounds and agents that can be used with the compositions of the present invention include, vitamins (e.g. D, E, A, K, and C), trace metals (e.g. zinc, calcium and selenium), anti-irritants (e.g. steroids and non-steroidal anti-inflammatories), botanical extracts (e.g. aloe vera, chamomile, cucumber extract, ginkgo biloba, ginseng, and rosemary), dyes and color ingredients (e.g. D&C blue no. 4, D&C green no. 5, D&C orange no. 4, D&C red no. 17, D&C red no. 33, D&C violet no. 2, D&C yellow no. 10, D&C yellow no. 11 and DEA-cetyl phosphate), emollients (i.e. organic esters, fatty acids, lanolin and its derivatives, plant and animal oils and fats, and di- and triglycerides), antimicrobial agents (e.g., triclosan and ethanol), and fragrances (natural and artificial).

J. Kits

In further embodiments of the invention, there is a provided a kit. Any of the compositions, compounds, agents, or active ingredients described in this specification may be comprised in a kit. In a non-limiting example, a kit can include a sunless tanning agent or a high pH composition, or both. The sunless tanning agent can be comprised in a composition or sunless tanning product.

The container means of the kits can include a bottle, dispenser, package, compartment, or other container means, into which a component may be placed. Where there is more than one component in the kit (they may be packaged together), the kit also will generally contain a second, third or other additional containers into which the additional components may be separately placed. The kits of the present invention also can include a means for containing the components in close confinement for commercial sale. Such containers may include injection or blow-molded plastic containers into which the desired bottles, dispensers, or packages are retained. For example, a kit of the present invention may include a container that has at least 2, 3, 4, 5, or more separated compartments. One compartment may include a high pH composition while the other compartment includes a sunless tanning composition. Alternatively, a kit may include separate containers where one container includes a high pH composition while a second container includes a sunless tanning composition.

A kit can also include instructions for employing the kit components as well the use of any other compositions, compounds, agents, active ingredients, or objects not included in the kit. Instructions may include variations that can be implemented. The instructions can include an explanation of how to apply, use, and maintain the products or compositions, for example.

### EXAMPLES

The following examples are included to demonstrate certain non-limiting aspects of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1

### Non-limiting Examples of a High pH Compositions

A non-limiting example of high pH compositions of the present invention are described in Tables 1 and 2. The pH of these compositions range from approximately 8.60-8.80. The ingredients in Tables 1 and 2 were formulated into skin moisturizing compositions.

TABLE 1
High pH moisturizing composition*

| | |
|---|---|
| Ingredient | % Concentration by weight |

| 1. | Oleic Acid | 6.4 |
|---|---|---|
| 2. | PEG-2 Stearate | 1.0 |
| 3. | Mineral Oil, Light | 5.2 |
| 4. | C10-30 Cholesterol | 0.75 |
| 5. | Propyl Paraben | 0.1 |
| 6. | Water | 66.17 |
| 7. | Xantham Gum | 0.2 |
| 8. | Glycerin 99.5% | 8.0 |
| 9. | Propylene Glycol | 2.2 |
| 10. | Disodium EDTA | 0.1 |
| 11. | Methyl Paraben | 0.3 |
| 12. | Sodium Hydroxide Sol | 9.58 |
| | Total | 100.0 |
| *Procedure used to prepare the high pH composition: Add ingredients 1-5 to an oil-phase vessel and heat to 70-75° C. while mixing. Add ingredient 6 to the main vessel and sprinkle in ingredient 7 while mixing. Mix for approximately 5 minutes and then | | |
| # begin heating and adding ingredients 8-11. Heat to 70-75° C. with mixing. Add the oil phase to the main vessel (water phase) at 70-75° C. and mix for 5 minutes. Add ingredient 12 and mix for 10 minutes. Begin slow cooling while mixing. Mix down to <30° C. | | |

TABLE 2
High pH moisturizing composition*

| | |
|---|---|
| Ingredient | % Concentration by weight |

| | | |
|---|---|---|
| 1. | Stearic Acid | 5.0 |
| 2. | Glyceryl monostearate | 0.55 |
| 3. | Cetyl Alcohol | 1.50 |
| 4. | Petrolatum | 3.00 |
| 5. | Dimethicone | 1.00 |
| 6. | Phenyltrimethicone | 0.75 |
| 7. | P-paraben | 0.10 |
| 8. | Mineral Oil | 4.00 |
| 9. | Water | 70.15 |
| 10. | Glycerin | 8.00 |
| 11. | Propylene Glycol | 2.00 |
| 12. | DiSodium EDTA | 0.10 |
| 13. | M-paraben | 0.20 |
| 14. | Sepigel 305 | 2.00 |
| | (polyacrylamide laureth 7, | |
| | C13-14 isoparraffin, water | |
| 15. | Triethanolamine (99%) | 1.60 |
| | Total | 100.0 |

*Procedure used to prepare the high pH composition: Add ingredients 9-13 into a main vessel and heat to 70-75° C. w/mixing. Heat ingredients 1-8 to 70-75 C. w/mixing. Add ingredients 1-8 to ingredients 9-13 at 70-75° C. w/mixing. Mix for 5 minutes, remove heat then add ingredient 14, mix for 10 minutes followed by the addition of ingredient 15. Cool with mixing to <30° C.

As noted above, the high pH compositions in Tables 1 and 2 are non-limiting examples only. It is contemplated that derivatives can be used as substitutes, additional compounds can be added, and compounds can be deleted from Tables 1 and 2. These and other aspects of the present invention are disclosed throughout this specification.

### Example 2

Testing Procedure Ten individuals were recruited for this study. At the initial visit a number of sites on the individuals' skin were marked on the volar forearm and the color was measured at each site with a Minolta Chromameter. This instrument provides 3 values—L*, which is a measure of light or dark; a* which is a measure of red; and b* which is a measure of yellow. Dihydroxyacetone (DHA), which is a known active ingredient in sunless tanning lotions, induces a yellow brown stain of the skin. This color change is observed as a change in the L* and b* values. Because the L* value can also be affected by other influences such as change in blood flow, only the b* value was evaluated for this study.

DHA (10% in a hydro-alcoholic gel) was applied under occlusion to each site (one control and four test sites). The panelists were instructed to remove these patches 6 hours later. Color measurements were again made the following morning (i.e., 24 hours after DHA application). This measurement was used as the baseline color. The panelists were given 1 oz. tubes of the high pH composition in Table 1. One site received no test material and served as an untreated control. The application of each product to a particular test site was randomized. Panelists were asked to apply the products each morning following cleansing and allow them to air dry. The panelists returned 2, 5, 7, and 9 days later for further color measurements. The change in brown color (b*) was determined as a percentage of the baseline b* value.

### Example 3

The efficacy of the composition in Table 1 in sunless tanning applications The efficacy of the composition in Table 1 to increase the duration of a sunless tan is described in Table 3.

TABLE 3
Effect of High pH Formulation on DHA Color
Percent Change from Baseline

| | Day 0 | Day 2 | Day 5 | Day 7 | Day 9 |
|---|---|---|---|---|---|
| Formula | b* | b* | B* | b* | b* |
| No Treatment | 100 | 112.21 | 91.99 | 87.24 | 53.46 |
| High pH | 100 | 128.56 | 114.15 | 108.89 | 88.48 |
| Composition (pH | | | | | |
| approximately | | | | | |
| 8.6-8.8) | | | | | |

These data indicate that there is an increase in color over the first 2 days for both the treated and untreated skin sites. The untreated skin site loses about half of its color approximately one week later. This can be a disadvantage in that it would increase the number of applications needed to maintain the sunless tan color. Application of the high pH composition causes a greater increase in the sunless tan color for the first days after application and maintains this above baseline levels for approximately one week. This can be advantageous in that it would decrease the number of applications needed to maintain the sunless tan color.

All of the compositions and/or methods disclosed and claimed in this specification can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 2,798,053
U.S. Pat. No. 3,755,560
U.S. Pat. No. 4,421,769
U.S. Pat. No. 4,509,949
U.S. Pat. No. 4,599,379
U.S. Pat. No. 4,628,078
U.S. Pat. No. 4,835,206
U.S. Pat. No. 4,849,484
U.S. Pat. No. 5,011,681
U.S. Pat. No. 5,087,445
U.S. Pat. No. 5,100,660
U.S. Pat. No. 5,559,146
U.S. Pat. No. 5,720,963
U.S. Pat. No. 6,262,541
U.S. Pat. No. 6,443,164
U.S. Pat. No. 6,447,760
U.S. Pat. No. 6,482,397
U.S. Pat. No. 6,495,126
Barany and Merrifield, In: The Peptides, Gross and Meienhofer (Eds.), Academic Press, NY, 1-284, 1979.
Houghten et al., Infect. Immun., 48(3):735-740, 1985.
International Cosmetic Ingredient Dictionary, 10th edition, 2004.
McCutcheon's, Detergents and Emulsifiers, North American Edition (1986).
Merrifield, Science, 232(4748):341-347, 1986.
Remington's Pharmaceutical Sciences, 18th Ed. Mack Printing Company, 1289-1329, 1990.
Schiltz et al. J. Investigative Dermatology, 87:663-667, 1986.
Stewart and Young, In: Solid Phase Peptide Synthesis, 2d. ed., Pierce Chemical Co., 1984.
Tam et al., J. Am. Chem. Soc., 105:6442, 1983.

1. A method for tanning skin comprising applying to the skin:
(a) a sunless tanning composition comprising dihydroxyacetone (DHA); and
(b) a high pH composition,
wherein the high pH composition is applied to the skin after the sunless tanning composition is applied to the skin.

2. The method of claim 1, wherein the high pH composition is applied to the skin at about 8 to about 14 hours after the sunless tanning composition.

3. The method of claim 1, wherein the sunless tanning composition and the high pH composition are comprised in separate compartments of a container.

4. The method of claim 1, wherein the sunless tanning composition and the high pH composition are comprised in separate containers.

5. The method of claim 1, wherein the high pH composition has a pH of about 7.5 to about 10.0.

6. The method of claim 5, wherein the high pH composition has a pH of about 8.0 to about 9.5.

7. The method of claim 1, wherein the high pH composition is further defined as a cleanser or moisturizer.

8. The method of claim 1, wherein the high pH composition comprises NaOH, KOH, or triethanol amine.

9. The method of claim 1, wherein the high pH composition comprises a fatty acid.

10. The method of claim 9, wherein the fatty acid is oleic acid or stearic acid.

11. The method of claim 1, wherein the pH composition is an emulsion or an anhydrous base.

12. The method of claim 11, wherein the pH composition is an emulsion and wherein the emulsion is a water-in-oil or an oil-in-water emulsion.

13. The method of claim 1, wherein the sunless tan lasts for at least about 7 to about 21 days.

14. A kit for artificially tanning skin comprising:
(a) a sunless tanning agent; and
(b) a high pH composition.

15. The kit of claim 14, wherein the sunless tanning agent and the high pH composition are comprised in separate containers.

16. The kit of claim 15, wherein the sunless tanning agent and the high pH composition are comprised in the same container.

17. The kit of claim 16, wherein the sunless tanning agent an the high pH compositions are comprised in separate compartments of the container.

18. The kit of claim 14, further comprising instructions.

19. A method for improving the effectiveness of a sunless tanning product comprising applying to skin:
(a) a sunless tanning product; and
(b) a high pH composition,
wherein the effectiveness of the sunless tanning product is improved by increasing the duration of a tan obtained by using the sunless tanning product.

20. The method of claim 19, wherein the sunless tanning product comprises dihydroxyacetone (DHA).
